(19) United States
(12) Patent Application Publication
(10) Pub. No.: US 2010/0099964 A1
(43) Pub. Date: Apr. 22, 2010

(54) HEMOGLOBIN MONITOR

(75) Inventors: Michael O'Reilly, Dan Point, CA (US); Ammar Al-Ali, Tustin, CA (US); Mohamed Diab, Ladera Ranch, CA (US); Paul Jansen, Cardiff by the Sea, CA (US); Arun Panch, Mission Viejo, CA (US)

Correspondence Address: KNOBBE MARTENS OLSON & BEAR LLP, 2040 MAIN STREET, FOURTEENTH FLOOR, IRVINE, CA, 92614, US

(73) Assignee: MASIMO Corporation, Irvine, CA (US)

(21) Appl. No.: 12/560,331

(22) Filed: Sep. 15, 2009

Related U.S. Application Data
(60) Provisional application No. 61/097,144, filed on Sep. 15, 2008.
(60) Provisional application No. 61/162,932, filed on Mar. 24, 2009.
(60) Provisional application No. 61/097,142, filed on Sep. 15, 2008.
(60) Provisional application No. 61/097,159, filed on Sep. 15, 2008.

(51) Int. Cl. A61B 5/1455; A61B 5/021

(52) U.S. Cl. 600/323; 600/485

(57) ABSTRACT

A patient monitor system is configured to measure and display a hemoglobin concentration measurement to assist caregivers in providing care or treatment and/or to automatically control a fluid, blood, medicine, or dialysis administration system. The patient monitor can analyze the displayed hemoglobin concentration measurement and provide alarms and feedback to assist caregivers. Additional measurement can be combined with the hemoglobin concentration measurement to provide combined displays helpful to caregivers, such as, for example, a plethysmograph variability index v. SpHb display.

### RELATED CASES

The present application claims priority benefit under 35 U.S.C. §119(e) from U.S. Provisional Application No. 61/097,144, filed Sep. 15, 2008, entitled, “System and Method for the Evaluation of a Patient's Condition;” U.S. Provisional Application No. 61/162,932, filed Mar. 24, 2009, entitled, “System and Method for the Evaluation of a Patient's Condition;” U.S. Provisional Application No. 61/097,142, filed Sep. 15, 2008, entitled, “Hemoglobin Medical Alarm;” and U.S. Provisional Application No. 61/097,159, filed Sep. 15, 2008, entitled, “Hemoglobin Correction and Alarm;” which are incorporated by reference herein.

### FIELD OF THE INVENTION

The present invention relates to the field of non-invasive determination, display, and alarm of a patient's physiological condition.

### BACKGROUND

Early detection of medical conditions of a patient is critical in improving the quality of care a patient receives. For example, early detection of internal bleeding and/or fluid responsiveness can generally be corrected if caught in time.

Knowing the composition of the patient's blood can provide significant information about the patient's condition, assist in patient diagnosis, and assist in determining a course of treatment. One blood component in particular, hemoglobin, is very important. Hemoglobin is responsible for the transport of oxygen from the lungs to the rest of the body. If there is insufficient total hemoglobin or if the hemoglobin is unable to bind with or carry enough oxygen, then the patient can suffocate. In addition to oxygen, other molecules can bind to hemoglobin. For example, hemoglobin can bind with carbon monoxide to form carboxyhemoglobin. When other molecules bind to hemoglobin, the hemoglobin is unable to carry oxygen molecules, and thus the patient is deprived of oxygen. Also, hemoglobin can change its molecular form and become unable to carry oxygen, this can occur, for example, with methemoglobin.

Standard monitoring devices, however, are unable to provide an indication of how much hemoglobin is in a patient's blood or whether other molecules were binding to hemoglobin and preventing the hemoglobin from binding with oxygen. Care givers generally measure parameters such as total hemoglobin, methemoglobin and carboxyhemoglobin by drawing blood and analyzing it in a lab. Given the nature of non-continuous blood analysis in a lab, it was widely believed that total hemoglobin did not change rapidly.

Advanced physiological monitoring systems utilize multiple wavelength sensors and multiple parameter monitors to provide enhanced measurement capabilities including, for example, the measurement of carboxyhemoglobin (HbCO), methemoglobin (HbMet) and total hemoglobin (SpHb™, Hbt or tHb). Physiological monitors and corresponding multiple wavelength optical sensors are described in at least U.S. Pat. Pub. No. 2006/0211924, filed Mar. 1, 2006 and titled Multiple Wavelength Sensor Emitters and U.S. Pat. Pub. No. 2006/0220881, filed Mar. 1, 2006 and titled Noninvasive Multi-Parameter Patient Monitor, both assigned to Masimo Laboratories, Irvine, Calif. (“Masimo Labs”) and both incorporated by reference herein. Commercially available patient monitors capable of measuring the above listed parameters is available from Masimo Corporation of Irvine, Calif.

### SUMMARY

The present disclosure discloses a system for utilizing patient monitors, such as pulse oximeters, for display and comparison of parameters which are useful in diagnosing the condition of a patient.

The present disclosure provides for the measurement, display and analysis of hemoglobin content in living patients. It has been discovered that, contrary to the widely held understanding that total hemoglobin does not change rapidly, total hemoglobin fluctuates over time. In an embodiment, the trend of a patient's continuous total hemoglobin (SpHb™, tHb or Hbt) measurement is displayed on a display. In an embodiment, the trend of the total hemoglobin is analyzed through, for example, a frequency domain analysis to determine patterns in the patient hemoglobin fluctuation. In an embodiment, a frequency domain analysis is used to determine a specific signature of the hemoglobin variability specific to a particular patient.

Additionally, exemplary uses of these hemoglobin readings are illustrated in conjunction with dialysis treatment and blood transfusions.

In an embodiment, the present disclosure discloses a total hemoglobin measurement system configured to provide an alarm that alerts a caregiver that a total hemoglobin measurement is outside of a specified range. In an embodiment, an alarm is activated when the hemoglobin measurement passes a high or low threshold. In an embodiment, the alarm is activated when the hemoglobin measurement is outside of a specified range for an amount of time. In an embodiment, an alarm is activated if a change in the hemoglobin measurement indicates that the measurement is likely to exceed a threshold.

In an embodiment, the present disclosure discloses a system for providing a correction between total hemoglobin measurements taken from venous blood and from arterial blood. Venous blood can have a different total hemoglobin amount then arterial blood. The difference in total hemoglobin between venous and arterial blood can be between about 0.1 and 2.5 g/dl or higher. In an embodiment, a system for measuring total hemoglobin is provided with an adjustment factor based on whether the system is measuring total hemoglobin from venous blood, arterial blood or both. In the situation where a measurement device, such as, for example, a pulse oximeter, is used to measure total hemoglobin and measures both a venous component and an arterial component, a correction factor can be applied to the outputted or displayed total hemoglobin levels to account for the difference in total hemoglobin between venous blood and arterial blood.

In an embodiment, multiple parameters measured by, or input to, a physiological measurement device are compared to provide a care giver with more information about the condition of the patient. This is important because a single parameter generally provides only limited information to a patient care giver. For example, a total hemoglobin measurement by itself can indicate a patient's hemoglobin concentrations. Similarly, a measurement of plethysmograph variability, central venous pressure (CVP), pulsus paradoxus, pulmonary capillary wedge pressure (PCWP), stroke volume variation, pulse pressure variation, systolic pressure variation (SPV), or the like, can indicate a patient's cardiac fluid responsiveness, volume status, hydration level or the like. However, when multiple parameters are compared with each other, new information becomes evident. For example, when Pleth Variability Index (PVI™) (or other similar fluid responsiveness measurements) and total hemoglobin (SpHb) are compared, information about whether a patient is bleeding or, in some cases, about to suffer heart failure can be provided. Similarly, when other parameters are compared, additional information can be provided which was previously unavailable.

In an embodiment, a graphical display illustrates the relationship between PVI and SpHb for a patient. In an embodiment, the display can indicate information about the PVI v. SpHb trend, such as, for example, the amount of time a PVI v. SpHb reading has remained substantially unchanged, the trend of the PVI v. SpHb, The direction in which the PVI v. SpHb readings are moving, etc. The display can also include alarms to alert a caregiver to a change in the patient's condition or to a PVI v. SpHb reading indicating that the patient requires treatment or that sufficient treatment has been received. Although described with respect to PVI, measurements of central venous pressure (CVP), pulsus paradoxus, pulmonary capillary wedge pressure (PCWP), stroke volume variation, pulse pressure variation, systolic pressure variation (SPV), or the like can be used instead of PVI or in addition to PVI.

In an embodiment, the PVI v. SpHb graph provides new information to a caregiver, previously unobtainable through other non-invasive measurement methods. In an embodiment, the PVI v. SpHb graph indicates whether a patient is hypovolemic or hypervolemic. In an embodiment, the PVI v. SpHb graph indicates whether the patient is bleeding. In an embodiment, the PVI v. SpHb graph indicates other physiological conditions such as heart failure. In an embodiment, the PVI v. SpHb graph indicates that a sufficient transfusion has been received. A caregiver can also use the provided information to determine a course of treatment to provide the best possible cardiac preload, for example, by administering more or less fluid.

In an embodiment, the device measuring the multiple physiological parameters can be used to indicate a recommended course of treatment. For example, the device can be used to visually or audible recommend a suggested course of treatment, such as, for example, administering more or less fluid, providing a blood transfusion, administering medicines, or the like. In an embodiment, the measurement device can be used in a system which automatically administers or adjusts fluid administration, blood transfusion, dialysis, or the like.

Although, by way of example, the present disclosure discusses comparing the parameters of PVI and SpHb, it is to be understood that any two parameters can be compared to provide additional information to a care giver. For example, any two or more of total hemoglobin, oxygen content, methemoglobin, carboxyhemoglobin, pleth variability index, oxyhemoglobin, perfusion index, pulse rate, blood pressure, or the like can be compared and displayed to the caregiver to provide additional information.

### BRIEF DESCRIPTION OF THE DRAWINGS

The drawings and following associated descriptions are provided to illustrate embodiments of the present disclosure and do not limit the scope of the claims. Corresponding numerals indicate corresponding parts, and the leading digit of each numbered item indicates the first figure in which an item is found.

FIG. 1 illustrates an example of a physiological monitoring system;

FIG. 2A illustrates a block diagram of an example prior art system for monitoring a patient's blood pressure and providing a blood transfusion;

FIGS. 2B and 2C illustrate block diagrams of embodiments of patient monitoring systems;

FIGS. 3A-3C illustrate embodiments of trend graphs of example hemoglobin values over time;

FIGS. 4A through 4C illustrate block diagrams of embodiments of fluid administration and/or transfusion control systems;

FIG. 4D illustrates a block diagram of an embodiment of a dialysis control process;

FIGS. 5 and 6 illustrate example physiological monitor displays;

FIG. 7 illustrates a block diagram of an embodiment of a patient monitoring system;

FIG. 8 illustrates an embodiment of a physiological measurement system with a PVI/SpHb display.

FIG. 9 illustrates an embodiment of a pulse oximeter screen with a PVI/SpHb display.

FIGS. 10A-I illustrate various possible PVI/SpHb displays for various potential conditions of a patient being monitored.

FIG. 11 illustrates an embodiment in which the boundaries between an acceptable reading and a non-acceptable reading are adjusted.

### DETAILED DESCRIPTION

Aspects of the disclosure will now be set forth in detail with respect to the figures and various embodiments. One of skill in the art will appreciate, however, that other embodiments and configurations of the devices and methods disclosed herein will still fall within the scope of this disclosure even if not described in the detail of some other embodiments. Aspects of various embodiments discussed do not limit the scope of the disclosure herein, which is instead defined by the claims following this description.

Some references that have common shorthand designations are referenced through such shorthand designations. For example, as used herein, HbCO designates carboxyhemoglobin, HbMet designates Methemoglobin and SpHb™ designates total hemoglobin. Other shorthand designations such as COHb, MetHb, and tHb or Hbt are also common in the art for these same constituents. These constituents are generally reported in terms of a percentage, often referred to as saturation, relative concentration or fractional saturation. Total hemoglobin is generally reported as a concentration in g/dL, g/L or millimoles (mMol). Other shorthand designations used herein include PI™, which designates perfusion index, and PVI™, which designates pleth variability index. Pleth variability index is generally reported as a percentage between 0% and 100%. The use of the particular shorthand designators presented in this application does not restrict the term to any particular manner in which the designated constituent is reported.

During and after surgery and in other care situations, blood pressure is a frequently monitored vital sign. For example, an anesthesiologist or other clinician may use blood pressure to determine whether to give fluid to a patient. If the patient's blood pressure drops, the clinician may determine that the patient needs increased fluid volume. To increase fluid volume, the clinician may provide blood or other volume-restoring fluid, such as a crystalloid or colloidal solution or the like. However, blood pressure alone does not indicate whether a patient needs blood or other volume-restoring fluid. Thus, out of an abundance of caution, clinicians may provide blood to patients instead of other volume-restoring fluid, even if the patients may not actually need blood. As a result, some patients are needlessly exposed to the risks of blood transfusions. Moreover, in some instances, patients may need a blood transfusion even when their blood pressure is relatively stable.

In an embodiment, this disclosure describes certain systems and methods for indicating when to provide blood, as opposed to volume-restoring fluid, to patients. In certain embodiments, a physiological monitor measures a patient's hemoglobin concentration noninvasively. The physiological monitor may indicate if the patient's hemoglobin concentration is low by generating a transfusion alarm or the like. A clinician may infer from the transfusion alarm or hemoglobin measurements that a blood transfusion, rather than volume-restoring fluid, is needed.

Noninvasively-measured hemoglobin may also be used in other medical situations, such as in dialysis treatment. A physiological monitor may measure a dialysis patient's hemoglobin concentration to determine whether too much blood is being taken from the patient. In such a situation, the physiological monitor can generate a dialysis alarm or the like. A clinician (or patient using dialysis at home) may infer from the dialysis alarm that dialysis treatment should be stopped or otherwise adjusted. Moreover, in alternative embodiments, a physiological monitor can directly control a transfusion or dialysis machine based on measured hemoglobin concentration.

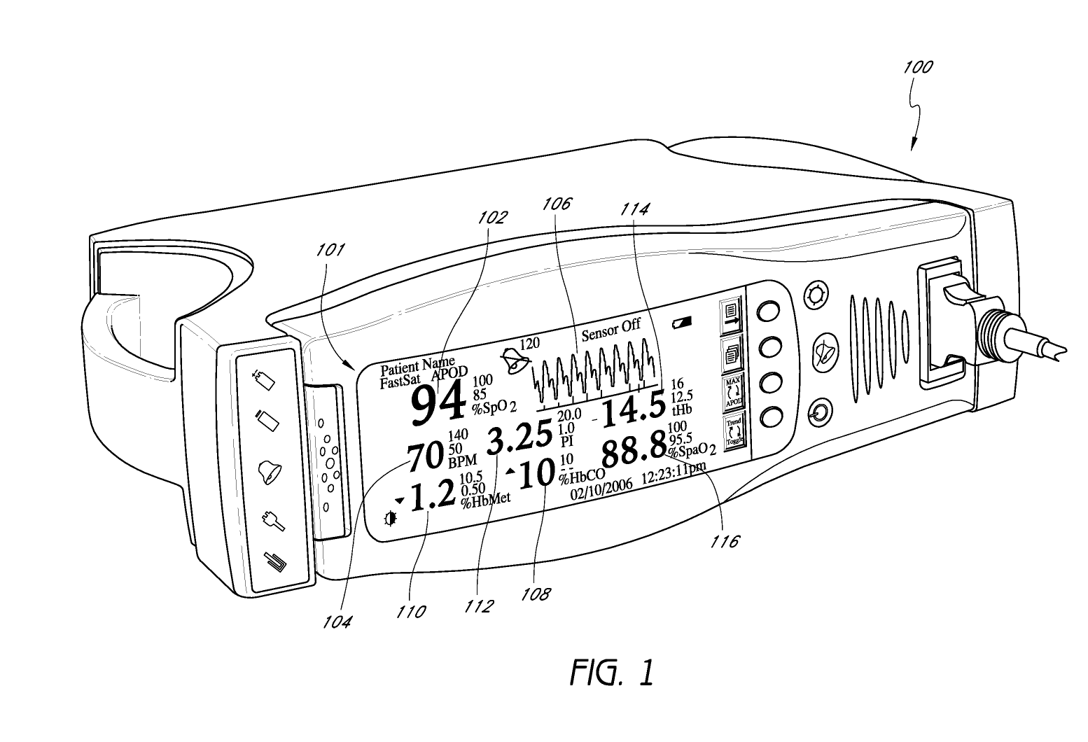

FIG. 1 illustrates an embodiment of a physiological monitor 100 configured to noninvasively measure hemoglobin concentration. The patient monitoring system 100 includes a patient monitor 102 attached to a sensor 106 by a cable 104. The sensor monitors various physiological data of a patient and sends signals indicative of the parameters to the patient monitor 102 for processing. The patient monitor 102 generally includes a display 108, control buttons 110, and a speaker 112 for audible alerts. The display 108 is capable of displaying readings of various monitored patient parameters, which may include numerical readouts, graphical readouts, and the like. Display 108 may be a liquid crystal display (LCD), a cathode ray tube (CRT), a plasma screen, a Light Emitting Diode (LED) screen, Organic Light Emitting Diode (OLED) screen, or any other suitable display. A patient monitoring system 102 may monitor oxygen saturation (SpO₂), perfusion index (PI), pulse rate (PR), hemoglobin count, and/or other parameters. An embodiment of a patient monitoring system according to the present disclosure is capable of measuring and displaying total hemoglobin trending data and preferably is capable of conducting data analysis as to the total hemoglobin trending.

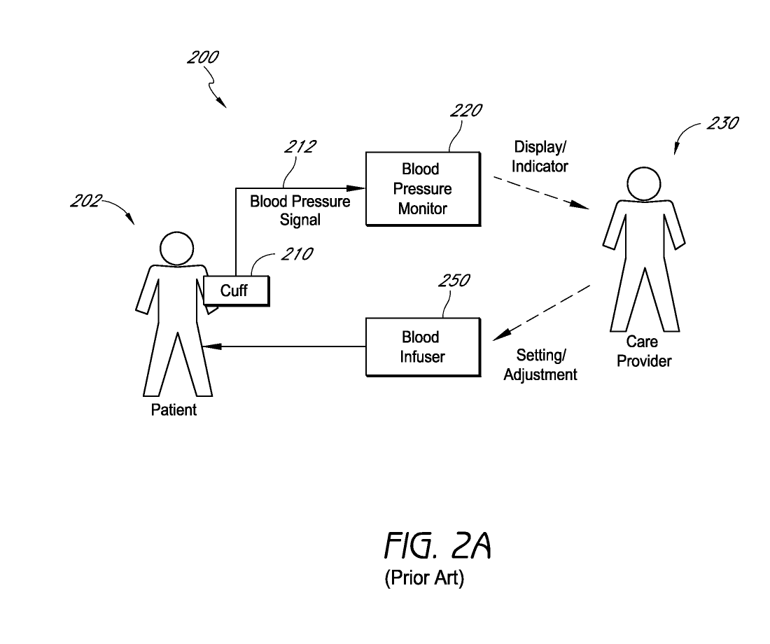

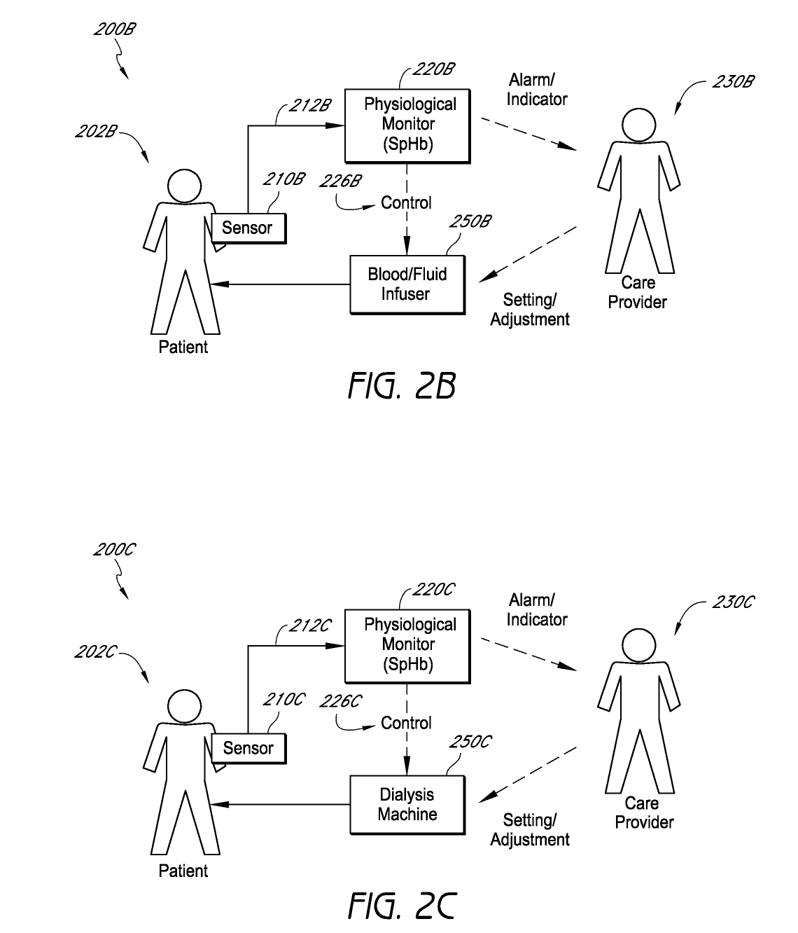

As described above, when clinicians determine whether a patient needs fluid volume, they often focus on the patient's blood pressure. For example, in FIG. 2, a prior art blood pressure monitoring system 200 is shown, which may be used in clinical settings such as in hospitals, ambulances, and the like. In the blood pressure monitoring system 200, a patient 202 is being monitored for blood pressure with a cuff 210 attached to the patient 202. The cuff 210 provides a blood pressure signal 212 to a blood pressure monitor 220. The blood pressure monitor 220 may be a traditional sphygmomanometer or an electronic blood pressure monitor.

The blood pressure monitor 220 outputs blood pressure values or a blood pressure indicator on a display viewed by a care provider 230. The care provider 230 may be a doctor, nurse, paramedic, technician, or the like. If the care provider 230 determines that the blood pressure values are too low, the care provider 130 may determine that the patient 202 needs blood. The care provider 230 may then provide blood to the patient 202 by adjusting settings of a blood infuser 250. The blood infuser 250 may then provide blood, such as donated blood from a blood bank, to the patient 202.

One drawback of using a blood pressure monitor 220 to determine when to give a patient 202 blood is that the blood pressure monitor 220 does not indicate when the patient 202 might need volume-restoring fluid instead of blood. The patient 202 may need volume-restoring fluid (crystalloid or colloidal solution or the like), for example, when the patient's 202 blood pressure is low but the patient's 202 hemoglobin concentration is within a normal range. Currently, the care provider 230 may not have access to the patient's 202 hemoglobin concentration without performing invasive blood gas measurements. As a precaution, the care provider 230 may therefore provide blood instead of volume-restoring fluid to the patient 202 even when the patient does not need blood.

In general, performing blood transfusions (including exchange transfusions) can present significant risks to the patient 202. The patient 202 may suffer a transfusion reaction, such as volume overload, iron overload, acute hemolytic reactions, anaphylactic reactions, and febrile non-hemolytic transfusion reactions, among others. Transfusion reactions can be exacerbated when the patient 202 receives unneeded blood.

To more accurately determine when to give a patient blood, a patient's hemoglobin concentration can be monitored. From the hemoglobin concentration, a clinician can infer when a blood transfusion may be needed.

FIGS. 2B and 2C illustrate example patient monitoring systems 200B and 200C, respectively. The patient monitoring systems 200 can be used to monitor the health status of a patient, including the status of a patient's hemoglobin. The patient monitoring system 200B can assist a care provider 230B with determining when to give a patient blood or fluid using, for example, the techniques described below. Likewise, the patient monitoring system 200C can assist a care provider 230C with determining when to adjust a patient's dialysis treatment using, for example, the techniques described above with respect to FIG. 2C.

Referring to FIG. 2B, a patient 202B is being monitored by a sensor 210B. The sensor 210B can be an optical sensor that irradiates a tissue site of the patient 202B with one or more wavelengths of electromagnetic radiation. The sensor 210B can detect radiation transmitted through the tissue site of the patient and provide an absorption signal 212B indicative of hemoglobin concentration to a physiological monitor 220B. The physiological monitor 220B can include one or more processors that can analyze the absorption signal 212B to determine one or more blood constituents of the patient 202B, such as hemoglobin concentration. Other examples of blood constituents that may be detected by the physiological monitor 220B are described below. In addition, more detailed embodiments of an example sensor and physiological monitor are also described below.

The physiological monitor 220B can output hemoglobin concentration values for display to a care provider 230B. In addition, the physiological monitor 220B can provide a hemoglobin trend graph, hemoglobin indicator, or the like that provides information on the patient's hemoglobin status. Moreover, the physiological monitor 220B may output an audio and/or visual alarm that recommends or otherwise indicates a desirability of providing a transfusion to the patient 202B. The physiological monitor 220B can use any of the methods described below in order to alert the care provider 230B as to the desirability of transfusing blood to the patient.

The care provider 230B can use the information provided by the physiological monitor 220B to determine whether to adjust settings 242B of a blood infuser 250B. The blood infuser 250B can be a device for performing blood transfusions, such as a rapid blood infuser. One example rapid blood infuser that can be used is the FMS 2000™, produced by the Belmont Instrument Corporation™.

In alternative embodiments, the physiological monitor 220B can provide a control signal 226B directly to the blood infuser 250B. The control signal 226B can cause the blood infuser 250B to transfuse blood to the patient. The physiological monitor 220B may generate this control signal 226B instead of or in addition to providing a trend graph, indicator, alarm, or the like. Similarly, although described with respect to blood infusion, an infusion of other liquids can also be controlled in the same way.

Referring to FIG. 2C, a patient 202C is connected to a sensor 210C, which can be an optical sensor, such as the sensor 210B described above with respect to FIG. 2B. The sensor 210C can monitor the patient's hemoglobin concentration, as well as other blood constituents, and provide an absorption signal 212C indicative of hemoglobin concentration to a physiological monitor 220C. The physiological monitor 220C can have some or all of the functionality of the physiological monitor 220B. For example, the physiological monitor 220C may analyze the absorption signal 212C to determine one or more blood constituents of the patient 202C, such as hemoglobin concentration.

The physiological monitor 220C can output hemoglobin concentration values for display to a care provider 230C. In addition, the physiological monitor 220C can provide a hemoglobin trend graph, hemoglobin indicator, or the like that provides information on the patient's hemoglobin status. Moreover, the physiological monitor 220C may output an audio and/or visual alarm that recommends or otherwise indicates a desirability of providing a transfusion to the patient 202C. The physiological monitor 220B can provide a trend graph, indicator, alarm, or the like to alert the care provider 230B as to the desirability of halting or reducing dialysis treatment. The care provider 230C may then make a setting or adjustment 242C to a dialysis machine 250C. In response to the setting or adjustment 242C from the care provider 230C, dialysis can be stopped or otherwise reduced.

In certain alternative embodiments, the physiological monitor 220C may send a control signal 226C to the dialysis machine 250C. The control signal 226C can adjust the dialysis treatment provided by the dialysis machine 250C. The physiological monitor 220C can generate this control signal 226C instead of or in addition to providing a trend graph, indicator, alarm, or the like.

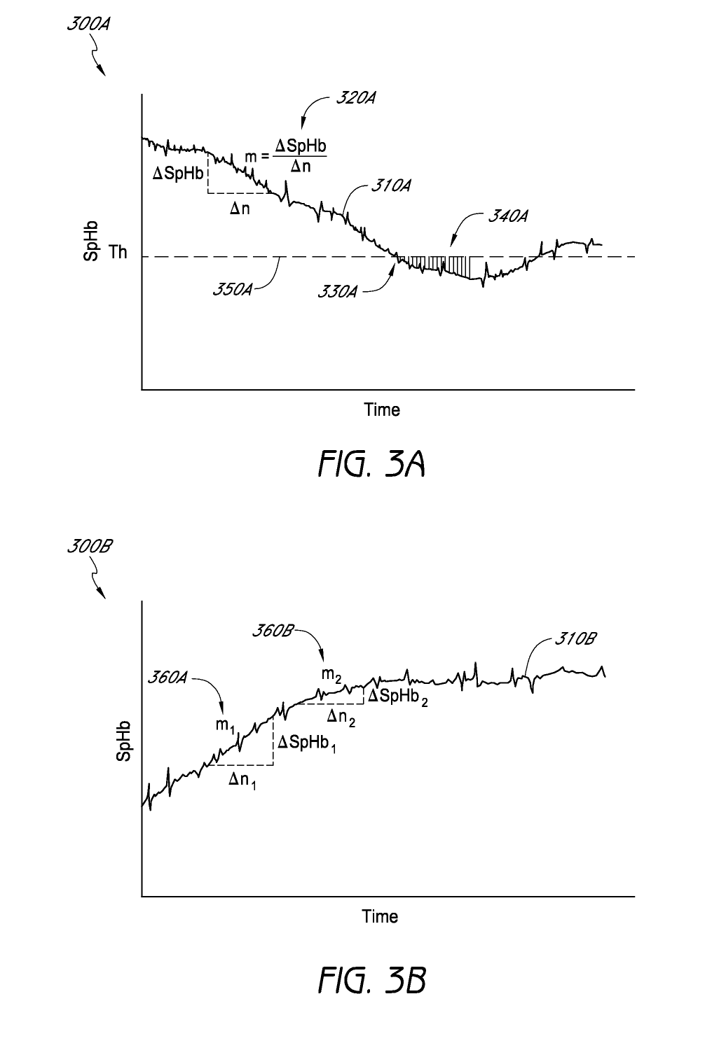

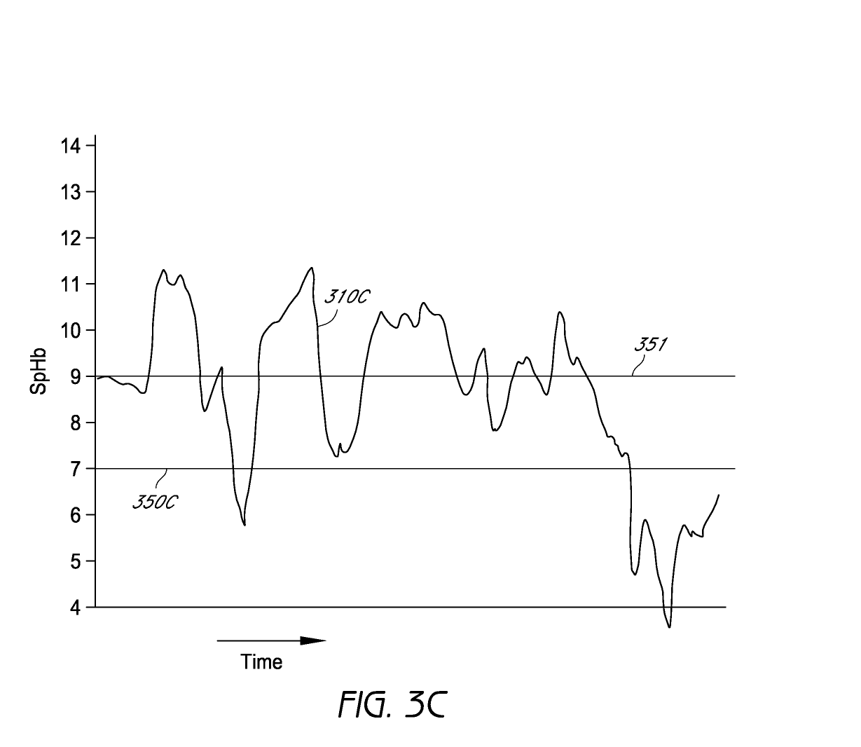

Referring to FIG. 3A, a hemoglobin trend graph 300A is shown that can assist clinicians in determining when to provide blood transfusions. The hemoglobin trend graph 300A depicts a hemoglobin curve 310A that illustrates an example patient's hemoglobin concentration over time. The hemoglobin measurements illustrated by the curve 310A can be obtained by a noninvasive physiological monitor as described herein. Several example techniques for determining when to perform a blood transfusion can be illustrated using the example hemoglobin curve 310A.

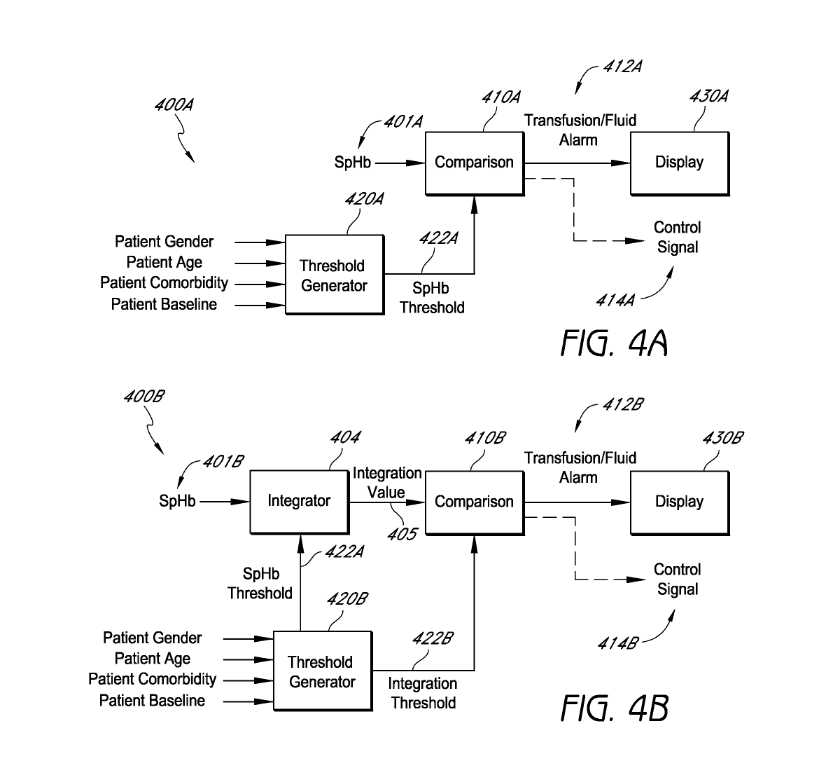

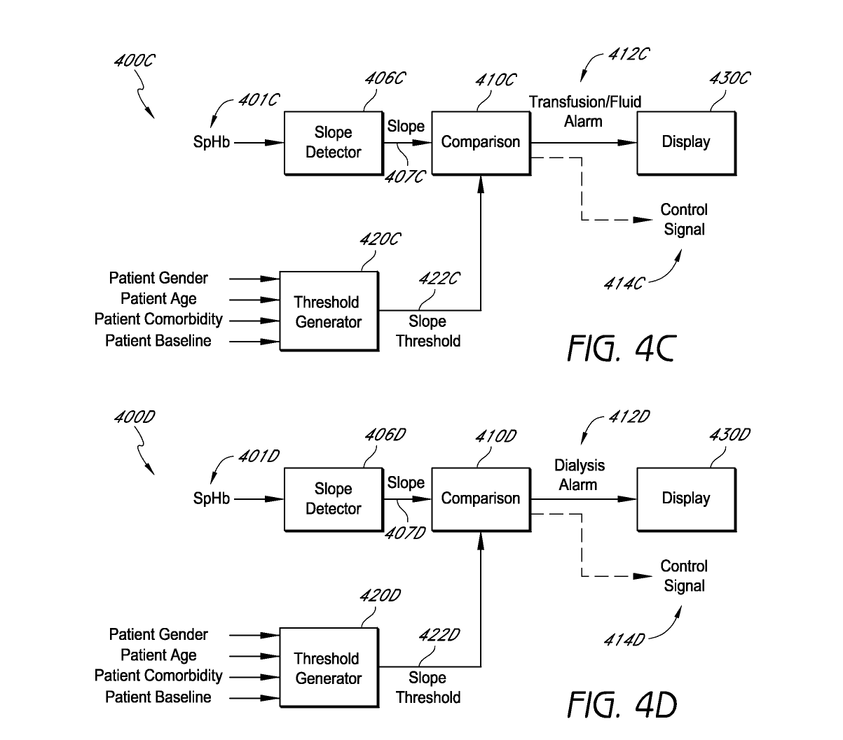

One technique can include determining when a patient's hemoglobin concentration goes above or below a threshold value. An example threshold 350A is shown superimposed on the hemoglobin trend graph 300A. Factors for determining the value of the threshold 350A are described below with respect to FIG. 4. If the patient's hemoglobin concentration, illustrated by the hemoglobin curve 310A, decreases below the threshold 350A (e.g., at point 330), then a care provider can decide to provide blood to the patient instead of volume-restoring fluid. On the other hand, if the hemoglobin curve 310A is above the threshold 350A but the patient is losing blood pressure, a care provider may decide to provide volume-restoring fluid to the patient instead of blood.

Thus, even if a patient's blood pressure is dropping but the hemoglobin is above the threshold 350A, a clinician may infer that the patient needs volume-restoring fluid instead of blood. On the other hand, if the blood pressure is stable, decreasing, or even increasing, but the hemoglobin is dropping below the threshold 350A, a clinician may decide to give the patient blood. The threshold 350A can therefore help clinicians make more informed decisions on whether to perform blood transfusions in certain embodiments.

The threshold 350A can be graphically depicted on a trend graph of a physiological monitor display. Thus, a care provider may be able to see that a patient's hemoglobin concentration has dropped below the threshold 350A and decide whether to provide blood. In addition, an audio and/or visual alarm can be generated when the hemoglobin curve 310A goes below the threshold 350A, indicating that a transfusion may be desired. Alternatively, in response to the hemoglobin concentration dropping below the threshold 350A, a physiological monitor can provide a control signal to a blood infuser (see FIG. 3). The control signal can directly cause the blood infuser to provide blood to the patient.

Hysteresis may be used with the threshold 350A to reduce false alarms. For example, hysteresis can be used to avoid generating an alarm when the patient's hemoglobin concentration oscillates about the threshold 350A, when the patient's hemoglobin concentration drops below the threshold 350A momentarily, or when the patient's hemoglobin concentration drops slightly below the threshold 350A for an extended period of time. Hysteresis can be provided by determining both an amount of time that a measured hemoglobin value has passed the threshold 350A and the amount by which the threshold 350A is passed. For example, the difference between hemoglobin values and the threshold 350A can be accumulated over time. The accumulation of hemoglobin values can be represented as an integral or integral approximation of an area between the hemoglobin curve 310A and the threshold 350A. A portion 340 of this area is represented on the hemoglobin trend graph 300A by darkened lines between the curve 310A and the threshold 350A. The integration value can be reset when the hemoglobin concentration returns to normal. Alternatively, the integration value can be gradually reset when the hemoglobin concentration is within normal range (e.g., above the threshold 350A) to provide additional hysteresis.

In some cases, it may be desirable to provide blood to a patient before the patient's hemoglobin concentration drops below the threshold 350A. This may be the case, for example, if the patient is losing blood quickly, such that the hemoglobin curve 310A may soon cross below the threshold 350A. Thus, another approach for determining when to perform a blood transfusion can be based upon detecting a slope 320 of the hemoglobin curve 310A. The slope 320A (m) of the hemoglobin curve 310A may be represented as a change in two or more hemoglobin concentration values (ΔSpHb) per change in sampled time (Δn, e.g., in seconds), or

$$m = \frac{Δ SpHb}{Δ n} . \qquad ( 1 )$$

In other embodiments, the slope 320A may be represented as a change between two average values of hemoglobin concentration per change in sampled time. The average values of hemoglobin may be used to reduce the effects of noise in the hemoglobin signal.

A negative value of this slope 320A can indicate that a patient's hemoglobin concentration is dropping. The more negative that the slope 320A is, the more likely it may be that the patient's hemoglobin concentration will drop below the threshold 350A. Thus, in certain embodiments a transfusion can be performed if the slope 320A exceeds a transfusion slope threshold (not shown). The transfusion slope threshold can be based on an experimentally-determined value, can vary based on the patient-dependent factors described below with respect to FIG. 4, can be a relative difference from a previous baseline slope value of the patient, combinations of the same, or the like. In alternative embodiments, a derivative or approximation of a derivative of the hemoglobin curve 310A may be used in place of the slope 320A. Alternatively, the slope 320A may be averaged over time and compared with the slope threshold. In addition, in some embodiments, the hemoglobin values represented by the hemoglobin curve 310A are averaged over time, smoothed or low pass filtered and the slope 320A is determined using the resulting hemoglobin values. Alternatively, threshold 350A can be an upper threshold or both an upper and lower threshold can be established.

As with the threshold 350A, the transfusion slope threshold may be graphically depicted on a trend graph of a physiological monitor display to assist a care provider in deciding when to provide blood to a patient. In addition, an audio and/or visual alarm can be generated when the transfusion slope threshold is exceeded. In alternative embodiments, a physiological monitor can provide a control signal to a blood infuser in response to the transfusion slope threshold being exceeded. The control signal can directly cause the blood infuser to provide blood to the patient.

Hysteresis can also be provided when the slope 320A is used so as to reduce false alarms. For example, hysteresis can be used to avoid providing transfusions when the slope of the hemoglobin curve 310A oscillates about the slope threshold, when the slope of the hemoglobin curve 310A drops below the slope threshold momentarily, or when the slope of the hemoglobin curve 310A drops slightly below the slope threshold for an extended period of time. Hysteresis is provided in certain embodiments by performing an integration or integration approximation of an area (not shown) between the slope 320A and the slope threshold. A transfusion alarm or indicator can be generated when the slope integration exceeds a threshold. The slope integration threshold can also be determined based on experimentation. In addition, the slope integration value can be gradually reset, or reset all at once, when the value returns above the threshold.

Although described mainly with respect to a lower threshold, an upper threshold in addition to, or as an alternative to the lower threshold 350A can also be used such that an alarm is generated when the hemoglobin curve 310A moves above the threshold in accordance with the above disclosure.

In an embodiment, the hemoglobin trend can also indicate a patient's volume status. The patient's volume status can be determined monitoring the hemoglobin concentration while administering fluid to the patient. A known quantity of fluid introduced can be compared with the change in hemoglobin concentration to determine the patient's volume status. This is because the introduced fluids dilute and lower the overall hemoglobin concentration. Thus, by knowing the amount of fluid introduced and the change in hemoglobin concentration, the patient's volume status can be determined.

As mentioned above, hemoglobin measurements can be used for purposes other than generating transfusion alarms, such as for augmenting dialysis treatment. Accordingly, FIG. 3B illustrates a hemoglobin trend graph 300B having a hemoglobin curve 310B of an example patient undergoing dialysis treatment. As above, the hemoglobin measurements illustrated by the curve 310B can be obtained by a physiological monitor as discussed above. From the hemoglobin curve 310B, a clinician (or patient using dialysis at home) may infer from the dialysis alarm that dialysis treatment should be stopped or otherwise adjusted. In an embodiment, the slope of the hemoglobin trend graph 300B during dialysis can indicate a patient's hemoglobin type (e.g. sickle cell anemia, normal, or the like). A patient's hemoglobin type can be can determined by comparing the slope of the hemoglobin trend during dialysis to empirically obtained data indicating the patient's hemoglobin type.

The hemoglobin curve 310B has a slope 360B. The slope 360B of the hemoglobin curve 310B is shown at two sections of the curve 310B, including an initial slope 360A at a first part of the curve 310B and a slope 360B at a second part of the curve 310B. The initial slope 360A indicates that the hemoglobin concentration is increasing at a rate m₁ (determined using an equation similar to equation (1) above). The hemoglobin concentration is increasing at the initial slope 360A because a dialysis machine is removing excess fluid from the patient's blood stream. Thus, a relatively steeper initial slope 360A can indicate a normal condition during dialysis.

At a later time in the hemoglobin trend graph 300B, the slope 360B of the hemoglobin curve 310B has a value m₂ (determined using an equation similar to equation (1) above) that is less than the value m₁ of the initial slope 360A. When the slope 360 of the hemoglobin curve 310B has decreased by a predetermined amount during dialysis, too much fluid may have been removed from the patient. When too much fluid is taken from the patient, the patient's body may react by drawing the interstitial fluid of the patient into the blood stream, thereby diluting the blood. As a result, the hemoglobin concentration of the blood can go down because it is diluted by the interstitial fluid. Thus, if the slope 360B of the hemoglobin curve 310B decreases below a certain value, such as a value relative to the initial slope 360A, it can be desirable to recommend a reduction or halting of the dialysis treatment to avoid further hemoglobin dilution.

The slope 360 at any point of the hemoglobin curve 310B can be compared with a dialysis slope threshold. The dialysis slope threshold can be based on an experimentally-determined value, can vary based on patient-dependent factors described below with respect to FIG. 4, can be a relative difference from a previous baseline slope value of the patient (for example, the initial slope 360A), combinations of the same, or the like. If the slope 360 drops below the dialysis slope threshold, a reduction or halting of dialysis treatment may be performed or an alarm can be activated to alert a patient or care giver to adjust to stop a dialysis treatment.

As above, the trend graph 300B can be displayed on a physiological monitor display, along with a dialysis slope threshold to indicate when dialysis adjustments may be desirable. An alarm can also be used to recommend adjustments to dialysis, or a control signal can be provided to a dialysis machine to control dialysis based on the slope 360 of the curve 310. In addition, in certain embodiments, the integration described with respect to FIG. 3A to provide hysteresis can also be applied to the dialysis slope threshold. In addition, in some embodiments, the hemoglobin values represented by the hemoglobin curve 310B are averaged, smoothed or low pass filtered, and the slope 360 is determined using the resulting hemoglobin values.

FIG. 3C illustrates a hemoglobin trend 310C during an organ transplant. An upper threshold 351 and a lower threshold 351C are provided to guide a caregiver. It can be desirable to keep the hemoglobin trend 310C between the upper and lower threshold 351 and 350C by, for example, adjusting blood transfusion and fluid administration, in order to keep the patient at appropriate levels. In an embodiment, a caregiver can adjust the thresholds. In an embodiment, the thresholds can be determined using experimentally determined values. In an embodiment, when a threshold is passed, an alarm can be activated. In an embodiment, hysteresis can be used to adjust when an alarm is activated to reduce unwanted alarms. In an embodiment, the patient monitoring device can be used to automatically initiate a blood transfusion or fluid administration.

FIGS. 4A through 4D illustrate example systems 400 for recommending transfusions or adjustments to dialysis treatment. Specifically, FIGS. 4A through 4C are directed toward systems 400A, 400B, and 400C for recommending transfusions, and FIG. 4D is directed toward a system 400D for recommending adjustments to dialysis treatment. The systems 400A through 400D can be implemented by any of the physiological monitors described herein. Each of the depicted blocks of the systems 400 represent hardware and/or software modules. While illustrated separately for purposes of description, the modules may share some or all of the same underlying hardware, logic, or code.

Referring to FIG. 4A, the system 400A receives a hemoglobin concentration (SpHb) 401A of a patient as an input to a comparison module 410A. A threshold generator 420A also provides a hemoglobin threshold 422A to the comparison module 410A. The threshold generator 420A can be a rules-based engine or the like that generates the threshold 422A using one or more factors such as patient gender, age, comorbidity, and patient baseline hemoglobin values.

For instance, if the patient is relatively healthy (e.g., little or no comorbidity), the threshold generator 420A can generate a hemoglobin threshold 422A of about 7 g/dL. If the patient has cardiac disease or other diseases (e.g., comorbidity), threshold generator 420A can generate a hemoglobin threshold 422A of about 10 g/dL. In addition, the threshold generator 420A can generate a higher hemoglobin threshold 422A if the patient's gender is male or lower if the patient's gender is female. The hemoglobin threshold 422A can be still lower if the patient is a pregnant female. In addition, if the patient is a child, the hemoglobin threshold 422A may be relatively lower than an adult's hemoglobin threshold 422A. Example normal hemoglobin ranges for various people include about 13.5-16.5 g/dL for healthy males, 12.1-15.1 g/dL for healthy females, 11-12 g/dL for pregnant women, and 11-16 g/dL for children. Moreover, the hemoglobin threshold 422A may take into account a patient's baseline hemoglobin concentration. For example, if the patient's baseline hemoglobin concentration is lower than average, the hemoglobin threshold 422A may be lower.

The comparison module 410A compares the hemoglobin concentration 401A of the patient with the hemoglobin threshold 422A. If the hemoglobin concentration 401A is below the hemoglobin threshold 422A, the comparison module 410A can output a transfusion alarm 412A to a display 430A, thus recommending to a care provider to perform a transfusion to the patient. The transfusion alarm 412A can be provided audibly instead of or in addition to being provided to the display 430A. In addition, the comparison module 410A can provide a control signal 414A to a blood infuser or the like. The control signal 414A can cause the blood infuser to provide blood to the patient and/or control an amount of blood provided to the patient. Alternatively, the above described system 400A can be used with respect to fluid infusion in addition to or as an alternative to a blood transfusion.

Referring to FIG. 4B, the system 400B receives a hemoglobin concentration 401B of a patient as an input to an integrator 404. A threshold generator 420B also provides the hemoglobin threshold 422A described above to the integrator 404. The integrator 404 can provide an integration value 405 to a comparison module 410B.

The threshold generator 420B can also provide an integration threshold 422B to the comparison module 410B. The integration threshold 422B can be determined experimentally and/or can be user-adjusted as described above. The comparison module 410B can compare the integration value 405 with the integration threshold 422B to determine if the integration threshold 422B has been exceeded. If so, the comparison module 410B can output a transfusion alarm 412B to a display 430B, recommending to a care provider 330B that a transfusion might be desirable. The transfusion alarm 412B may be provided audibly instead of or in addition to being provided to the display 430B. In addition, the comparison module 410B can provide a control signal 414B directly to a blood transfusion device, causing the blood transfusion device to provide a blood transfusion to the patient. Alternatively, the above described system 400B can be used with respect to fluid infusion in addition to or as an alternative to a blood transfusion.

Referring to FIG. 4C, the system 400C receives a hemoglobin concentration 401C as an input to a slope detector 406C. The slope detector 406C outputs a slope 407C of the hemoglobin values. The calculation of the slope 407C is described above. The slope 407C is provided to a comparison module 410C. A slope threshold 422C is also provided to the comparison module 410C from a threshold generator 420C. The slope threshold 422C can be generated experimentally, can vary based on the patient-dependent factors described above with respect to FIG. 4A, can be a relative difference from a previous baseline slope value of the patient, combinations of the same, or the like.

The comparison module 410C can compare the slope 407C with the slope threshold 422C to determine if the slope threshold 422C has been exceeded. If so, the comparison module 410C can output a transfusion alarm 412C to a display 430C and/or to an audible device. In addition, the comparison module 410C can provide a control signal 414C directly to a blood transfusion device as described above. Alternatively, the above described system 400C can be used with respect to fluid infusion in addition to or as an alternative to a blood transfusion.

Referring to FIG. 4D, the system 400D receives a hemoglobin concentration 401 D as an input to a slope detector 406D. The slope detector 406D outputs a slope 407D of the hemoglobin values. The calculation of the slope 407D is described above. The slope 407D is provided to a comparison module 410D. A slope threshold 422D is also provided to the comparison module 410D from a threshold generator 420D. The slope threshold 422D can be generated experimentally, can vary based on the patient-dependent factors described below with respect to FIG. 4A, can be a relative difference from a previous baseline slope value of the patient, combinations of the same, or the like.

The comparison module 410D can compare the slope 407d with the slope threshold 422D to determine if the slope threshold 422D has been exceeded. If so, the comparison module 410D can output a dialysis alarm 412D to a display 430D and/or to an audible device, recommending an adjustment in dialysis treatment to a care provider. Alternatively, the comparison module 410D can provide a control signal 414D directly to a dialysis machine.

Although described mainly with respect to fluid administration, blood transfusion and dialysis treatment, the above described processes, determinations and alarms can also be equally applied to drug delivery and other administration procedures as would be understood by those of skill in the art. For example, diuretics are often administered to treat congestive heart failure. But it can be difficult to determine an optimal amount of diuretics to administer. The total hemoglobin concentration by itself or in conjunction with other parameters (such as described below) can be used to determine the optimal diuretics dosage to administer. Because diuretics reduce the amount of fluids in the blood stream, a caregiver can analyze the rise in hemoglobin concentration in order to provide an optimal dose. Similarly, using hemoglobin concentration in conjunction with a volume status indicator, such as PVI (as described below) can further assist in the administration of diuretics by providing information on the patient's heart preload as it is affected by the diuretics.

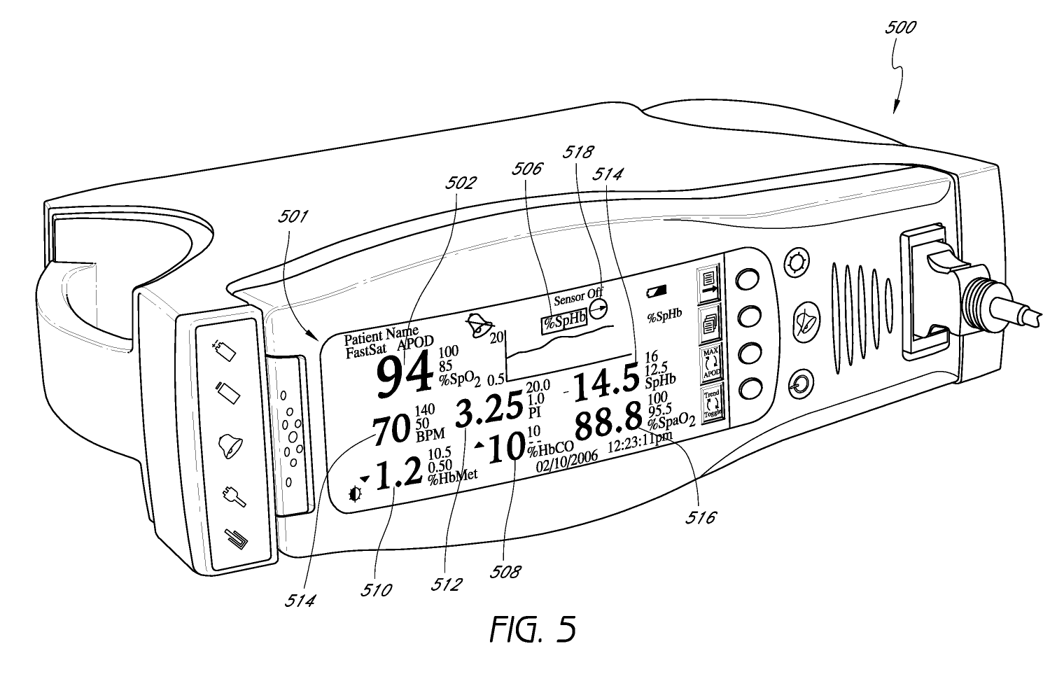

FIG. 5 illustrates an example display of an example noninvasive multi-parameter physiological monitor 500. An embodiment of the monitor 500 includes a display 501 showing a plurality of parameter data. For example, the display 501 can advantageously include a display, such as a CRT or an LCD display including circuitry similar to that available on patient care devices commercially available, such as, for example, from Masimo Corporation of Irvine, Calif. sold under the name Radical™, and disclosed in the U.S. patents referenced above and incorporated above. Many other commercially available display components can be used that are capable of displaying hemoglobin concentration and other physiological parameter data along with the ability to display graphical data such as plethysmographs, trend graphs or traces, and the like.

The depicted embodiment of the display 501 includes a measured value of SpHb 514 and an SpHb trend graph 506. In addition, other measured blood constituents shown include SpO₂ 502, pulse rate 504 in beats per minute (BPM), HbCO 508, HbMet 510, perfusion quality 512, and a derived value of fractional saturation “SpaO₂” 516. Many other blood constituents or other physiological parameters can be measured and displayed by the physiological monitor 500, such as blood pressure, ECG readings, acoustic respiratory measurements, and the like. Alternatively, other physiological measurement devices can be used to measure a desired parameter which can then be input to the the physiological monitor 500.

A hemoglobin indicator 518 is also depicted, which shows the current slope trend for SpHb. The hemoglobin indicator 518 includes an arrow that can point in a direction of a slope of the SpHb trend graph 506. A care provider can make decisions based on the slope of the hemoglobin indicator 518, such as whether to start or adjust a blood transfusion or to stop or adjust dialysis treatment. The hemoglobin indicator 518 can also function as an alarm by flashing, changing color, or the like when the slope of the SpHb trend graph 506 is too high or too low. The hemoglobin indicator 518 can alarm in order to signal a recommended blood transfusion and/or to signal a recommended adjustment in dialysis treatment.

In alternative embodiments, the hemoglobin indicator 518 can instead include an up arrow, a down arrow, and a hyphen bar to indicate up trending/prediction, down trending/prediction, or neutral trending/prediction. Many other variations on the hemoglobin indicator 518 are also possible, such as text (e.g., “Low SpHb” and the like), colors (e.g., red, yellow, and green), combinations of the same, and the like.

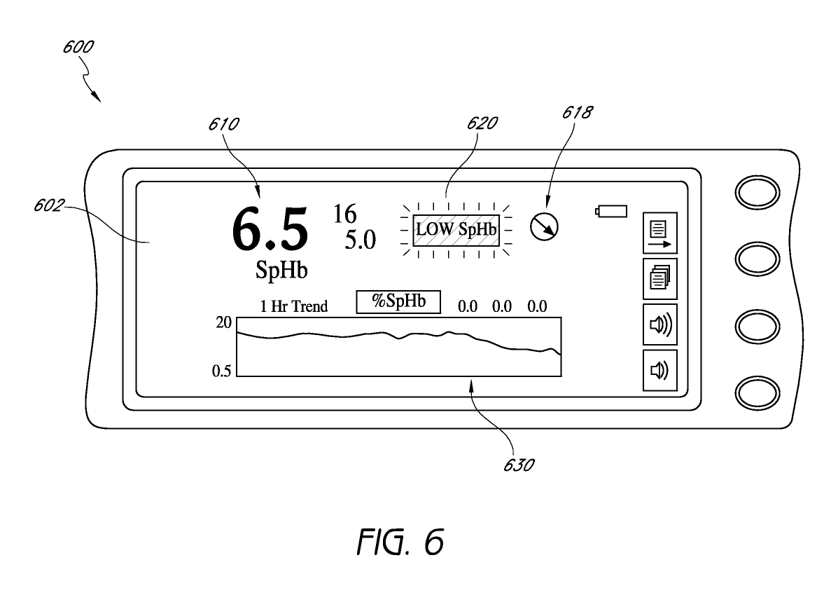

FIG. 6 illustrates another example physiological monitor 600 having a display 602. The display 602 includes parameter data for hemoglobin, including a measured value of SpHb 610, a SpHb trend graph 630, and a hemoglobin indicator 618. The hemoglobin indicator 618 can have the same functionality of the hemoglobin indicator 518 described above. For example, the hemoglobin indicator 618 can function as an alarm in certain embodiments by flashing, changing color, or the like when the slope of the SpHb trend graph 630 is too high or too low.

A visual hemoglobin alarm 620 is also shown. The depicted embodiment of the visual hemoglobin alarm hemoglobin alarm 620 includes text that indicates that the SpHb concentration is low. The visual hemoglobin alarm 620 can be displayed, for example, when a patient's hemoglobin concentration drops below a threshold value, when the slope of the SpHb trend graph 630 decreases below a slope threshold, when an integrated value exceeds an integration threshold as described above, combinations of the same, or the like. The visual hemoglobin alarm 620 can be accompanied by or replaced by an audio alarm in certain embodiments. The visual hemoglobin alarm 620 and/or audible alarm can indicate to a care provider that a blood transfusion is desired.

While the visual alarm 620 includes the text “Low SpHb,” this text can be different based on whether the alarm 620 is generated in response to a low threshold, drop in slope, or the like. The text of the alarm 620 can also reference a desired transfusion, such as “Transfuse Blood.” In addition, the alarm 620 need not have text at all but can have some other visual indicator of low SpHb or a recommended transfusion. In addition, while not shown, the visual hemoglobin alarm 620 can also indicate that an adjustment to dialysis treatment may be desired in certain embodiments.

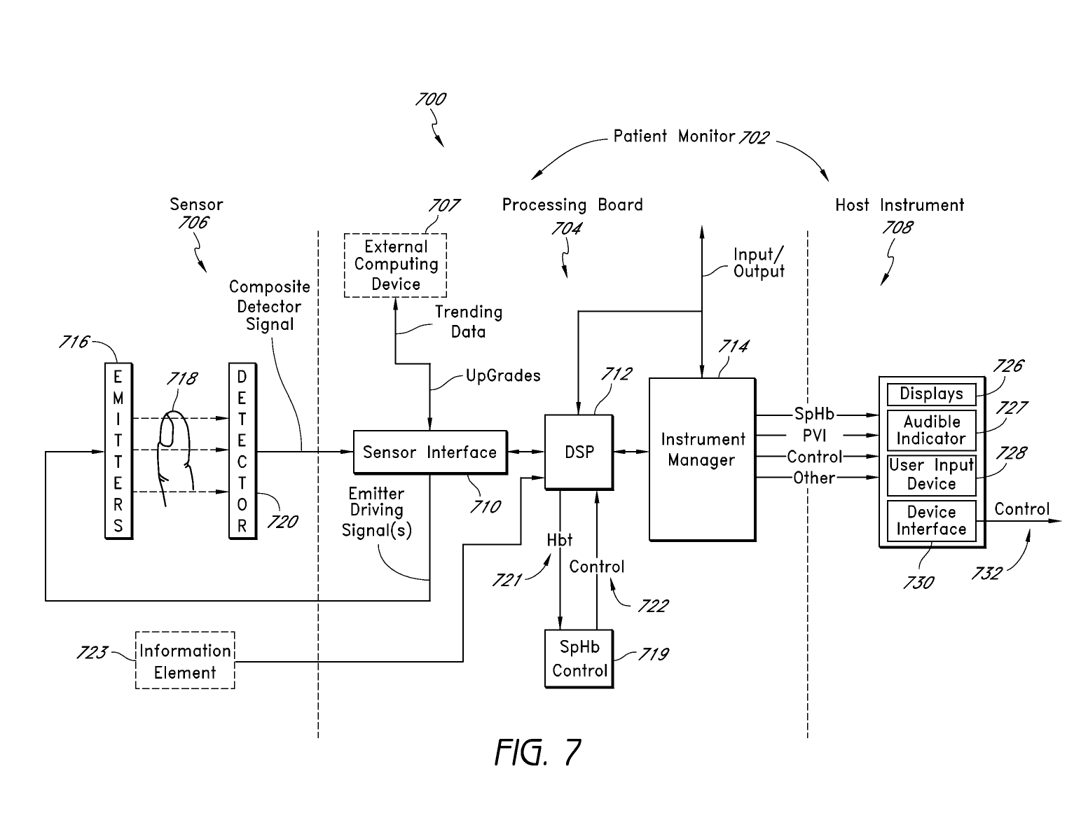

FIG. 7 illustrates an embodiment of a patient monitoring system that can implement any of the systems described herein. FIG. 7 illustrates a block diagram of an exemplary embodiment of a patient monitoring system 700. As shown in FIG. 7, the system 700 includes a patient monitor 702 including a processing board 704 and a host instrument 708. The processing board 704 communicates with a sensor 706 to receive one or more intensity signal(s) indicative of one or more parameters of tissue of a patient. The processing board 704 also communicates with a host instrument 708 to display determined values calculated using the one or more intensity signals. According to an embodiment, the board 704 comprises processing circuitry arranged on one or more printed circuit boards capable of installation into the monitor 702, or capable of being distributed as some or all of one or more OEM components for a wide variety of host instruments monitoring a wide variety of patient information. In an embodiment, the processing board 702 comprises a sensor interface 710, a digital signal processor and signal extractor (“DSP” or “processor”) 712, and an instrument manager 714. In general, the sensor interface 710 converts digital control signals into analog drive signals capable of driving sensor emitters, and converts composite analog intensity signal(s) from light sensitive detectors into digital data.

In an embodiment, the sensor interface 710 manages communication with external computing devices. For example, in an embodiment, a multipurpose sensor port (or input/output port) is capable of connecting to the sensor 706 or alternatively connecting to a computing device, such as a personal computer, a PDA, additional monitoring equipment or networks, or the like. When connected to the computing device, the processing board 704 can upload various stored data for, for example, off-line analysis and diagnosis. The stored data can comprise trend data for any one or more of the measured parameter data, plethysmograph waveform data acoustic sound waveform, or the like. Moreover, the processing board 704 can advantageously download from the computing device various upgrades or executable programs, can perform diagnosis on the hardware or software of the monitor 702. In addition, the processing board 704 can advantageously be used to view and examine patient data, including raw data, at or away from a monitoring site, through data uploads/downloads, or network connections, combinations, or the like, such as for customer support purposes including software maintenance, customer technical support, and the like. Upgradable sensor ports are disclosed in copending U.S. application Ser. No. 10/898,680, filed on Jul. 23, 2004, titled “Multipurpose Sensor Port,” incorporated by reference herein.

As shown in FIG. 7, the digital data is output to the DSP 712. According to an embodiment, the DSP 712 comprises a processing device based on the Super Harvard ARChitecture (“SHARC”), such as those commercially available from Analog Devices. However, the DSP 712 can also comprise a wide variety of data and/or signal processors capable of executing programs for determining physiological parameters from input data. In particular, the DSP 712 can include program instructions capable of receiving multiple channels of data related to one or more intensity signals representative of the absorption (from transmissive or reflective sensor systems) of a plurality of wavelengths of emitted light by body tissue. In an embodiment, the DSP 712 accepts data related to the absorption of eight (8) wavelengths of light, although the data can be related to the absorption of two (2) to sixteen (16) or more wavelengths.

The DSP 712 can also communicate with an SpHb control process 719, which can include firmware stored in a memory device (not shown). The SpHb control process 719 can run on the DSP 712 or a separate DSP. The SpHb control process 719 can receive SpHb values 721 and generate a control signal 722 that is communicated directly or indirectly to a device interface 730. The device interface 730 can be part of the host instrument 708 and can interface with a blood infuser, dialysis machine, or the like. The device interface 730 can provide a corresponding control signal 732 to a blood infuser to control an amount of blood infused into a patient or to a dialysis machine to control dialysis treatment.

FIG. 7 also shows the processing board 704 including the instrument manager 714. According to an embodiment, the instrument manager 714 can comprise one or more microcontrollers controlling system management, including, for example, communications of calculated parameter data and the like to the host instrument 708. The instrument manager 714 can also act as a watchdog circuit by, for example, monitoring the activity of the DSP 712 and resetting it when appropriate.

The sensor 706 can include a reusable clip-type sensor, a disposable adhesive-type sensor, a combination sensor having reusable and disposable components, or the like. Moreover, the sensor 706 can also comprise mechanical structures, adhesive or other tape structures, Velcro™ wraps or combination structures specialized for the type of patient, type of monitoring, type of monitor, or the like. In an embodiment, the sensor 706 provides data to the board 704 and vice versa through, for example, a patient cable. Such communication can be wireless, over public or private networks or computing systems or devices, or the like.

As shown in FIG. 7, the sensor 706 includes a plurality of emitters 716 irradiating the body tissue 718 with differing wavelengths of light, and one or more detectors 720 capable of detecting the light after attenuation by the tissue 718. In an embodiment, the emitters 716 include a matrix of eight (8) emission devices mounted on a flexible substrate, the emission devices capable of emitting eight (8) differing wavelengths of light. In other embodiments, the emitters 716 can include twelve (12) or sixteen (16) emitters, although other numbers of emitters are contemplated, including two (2) or more emitters. As shown in FIG. 7, the sensor 706 can include other electrical components such as, for example, an information element 723 that can be a memory device comprising an EPROM, EEPROM, ROM, RAM, microcontroller, combinations of the same, or the like. In an embodiment, other sensor components can include a temperature determination device (not shown) or other mechanisms for, for example, determining real-time emission wavelengths of the emitters 716.

The information element 723 in certain embodiments advantageously stores some or all of a wide variety data and information, including, for example, information on the type or operation of the sensor 706, type or identification of sensor buyer or distributor or groups of buyer or distributors, sensor manufacturer information, sensor characteristics including the number of emitting devices, the number of emission wavelengths, data relating to emission centroids, data relating to a change in emission characteristics based on varying temperature, history of the sensor temperature, current, or voltage, emitter specifications, emitter drive requirements, demodulation data, calculation mode data, the parameters for which the sensor is capable of supplying sufficient measurement data (e.g., HbCO, HbMet, SpHb, or the like), calibration or parameter coefficient data, software such as scripts, executable code, or the like, sensor electronic elements, whether the sensor is a disposable, reusable, multi-site, partially reusable, partially disposable sensor, whether it is an adhesive or non-adhesive sensor, whether the sensor is a reflectance, transmittance, or transreflectance sensor, whether the sensor is a finger, hand, foot, forehead, or ear sensor, whether the sensor is a stereo sensor or a two-headed sensor, sensor life data indicating whether some or all sensor components have expired and should be replaced, encryption information, keys, indexes to keys or hash functions, or the like, monitor or algorithm upgrade instructions or data, some or all of parameter equations, information about the patient, age, gender, medications, comorbidity, and other information that may be useful for the accuracy or alarm settings and sensitivities, trend history, alarm history, or the like. In certain embodiments, the monitor can advantageously store data on the memory device, including, for example, measured trending data for any number of parameters for any number of patients, or the like, sensor use or expiration calculations, sensor history, or the like.

FIG. 7 also shows the patient monitor 702 including the host instrument 708. In an embodiment, the host instrument 708 communicates with the board 704 to receive signals indicative of the physiological parameter information calculated by the DSP 712. The host instrument 708 preferably includes one or more display devices 726 capable of displaying indicia representative of the calculated physiological parameters of the tissue 718 at the measurement site. In an embodiment, the host instrument 708 can advantageously comprise a handheld housing capable of displaying SpHb and one or more other parameters such as pulse rate, plethysmograph data, perfusion quality such as a perfusion quality index (“PI™”), signal or measurement quality (“SQ”), values of blood constituents in body tissue, including for example, SpO₂, HbCO, HbMet, or the like. In other embodiments, the host instrument 708 is capable of displaying values for one or more of blood glucose, bilirubin, or the like. The host instrument 708 can be capable of storing or displaying historical or trending data related to one or more of the measured values, combinations of the measured values, plethysmograph data, or the like. The host instrument 708 also includes an audio indicator 727 and user input device 728, such as, for example, a keypad, touch screen, pointing device, voice recognition device, or the like.

In still additional embodiments, the host instrument 708 includes audio or visual alarms that alert caregivers that one or more physiological parameters are falling below predetermined safe thresholds. The host instrument 708 can include indications of the confidence a caregiver should have in the displayed data. In a further embodiment, the host instrument 708 can advantageously include circuitry capable of determining the expiration or overuse of components of the sensor 706, including, for example, reusable elements, disposable elements, or combinations of the same.

Although the present disclosure discusses a non-invasive continuous measurement of total hemoglobin, the present disclosure is equally applicable to invasive and non-continuous measurements of total hemoglobin.

Moreover, in an embodiment, a physiological measurement system provides a correction between total hemoglobin measurements taken from venous blood and from arterial blood. Venous blood can have a different total hemoglobin level than arterial blood. Often, venous blood has a higher total hemoglobin then arterial blood does. The difference in total hemoglobin between venous and arterial blood can be between about 0.1 and 2.5 g/dl and higher. The difference can change based on the patient and the patient's current condition. The difference can also change over time for the same patient. In an embodiment, a system for measuring total hemoglobin is provided with an adjustment factor based on the whether the system is measuring total hemoglobin from venous blood or arterial blood. In the situation where a measurement device, such as, for example, a pulse oximeter, is used to measure total hemoglobin and measures both a venous component and an arterial component, a correction factor can be applied to the outputted or displayed total hemoglobin levels to account for the difference between in total hemoglobin between venous blood and arterial blood.

In an embodiment, the difference between total hemoglobin measurements between venous and arterial blood is used to provide a more accurate measurement of total hemoglobin. In an embodiment, noninvasive measurements of total hemoglobin are generated based on a model of the patient's tissue. For example, in pulse oximetry, ratios of attenuated light of different wavelengths are used to cancel out constant tissue attenuation factors such as skin and bone. However, both venous and arterial blood affect measurements of total hemoglobin. Using the knowledge that venous and arterial blood may have different total hemoglobin levels can be used in the model to provide for a more accurate measurement of total hemoglobin. For example, if arterial total hemoglobin measurements are desired, the actual measured total hemoglobin amount can be adjusted down to account for the differences in total hemoglobin levels.

In an embodiment, a correction factor option is provided on a device that measures total hemoglobin. The correction factor can be provided to both a noninvasive or invasive total hemoglobin monitor as well as a continuous or non-continuous total hemoglobin monitor. In an embodiment, the correction factor is determined by taking a measurement of total hemoglobin in a vein and a measurement of total hemoglobin in an artery. In an embodiment, the correction factor can be based on empirically obtained data from a large sample of patients to provide a predicted correction factor. In an embodiment, the correction factor option is provided in either software or hardware or both. The correction factor option can be a switch or button on the device to indicate whether a measurement of venous or arterial blood or both is being measured. Similarly, the correction factor can be a software function which provides a similar option to the caregiver.

In an embodiment, a measurement of total hemoglobin for both arterial and venous blood is measured. The measurement can be a graph, trend or instantaneous measurement. In an embodiment, the measurements are compared and a trend illustrating the difference in total hemoglobin for arterial and venous blood can be determined. The comparison measurement can be used to determine a condition of the patient. For example, if the comparison shows a increasing or decreasing divergence in total hemoglobin, the monitor can provide an alarm to a caregiver to alert the caregiver to a condition of the patient.

In an embodiment, the hemoglobin measurement can be calibrated by taking invasive measurements and inputting invasive measurement determinations into the device to adjust the non-invasive measurement.

Although described in terms of certain embodiments, other embodiments can also be provided. For example, the monitor 702 can include one or more monitoring systems monitoring parameters, such as, for example, vital signs, blood pressure, ECG or EKG, respiration, glucose, bilirubin, or the like. Such systems can combine other information with intensity-derived information to influence diagnosis or device operation. Moreover, the monitor 702 can advantageously include an audio system, preferably comprising a high quality audio processor and high quality speakers to provide for voiced alarms, messaging, or the like. In an embodiment, the monitor 702 can advantageously include an audio out jack, conventional audio jacks, headphone jacks, or the like, such that any of the display information disclosed herein may be audiblized for a listener. For example, the monitor 702 can include an audible transducer input (such as a microphone, piezoelectric sensor, or the like) for collecting one or more of heart sounds, lung sounds, trachea sounds, or other body sounds and such sounds may be reproduced through the audio system and output from the monitor 702. Also, wired or wireless communications (such as Bluetooth or WiFi, including IEEE 801.11a, b, g, n, or the like), mobile communications, combinations of the same, or the like, may be used to transmit the audio output to other audio transducers separate from the monitor 1702.

For example, patterns or changes in the continuous noninvasive monitoring of intensity-derived information can cause the activation of other vital sign measurement devices, such as, for example, blood pressure cuffs.

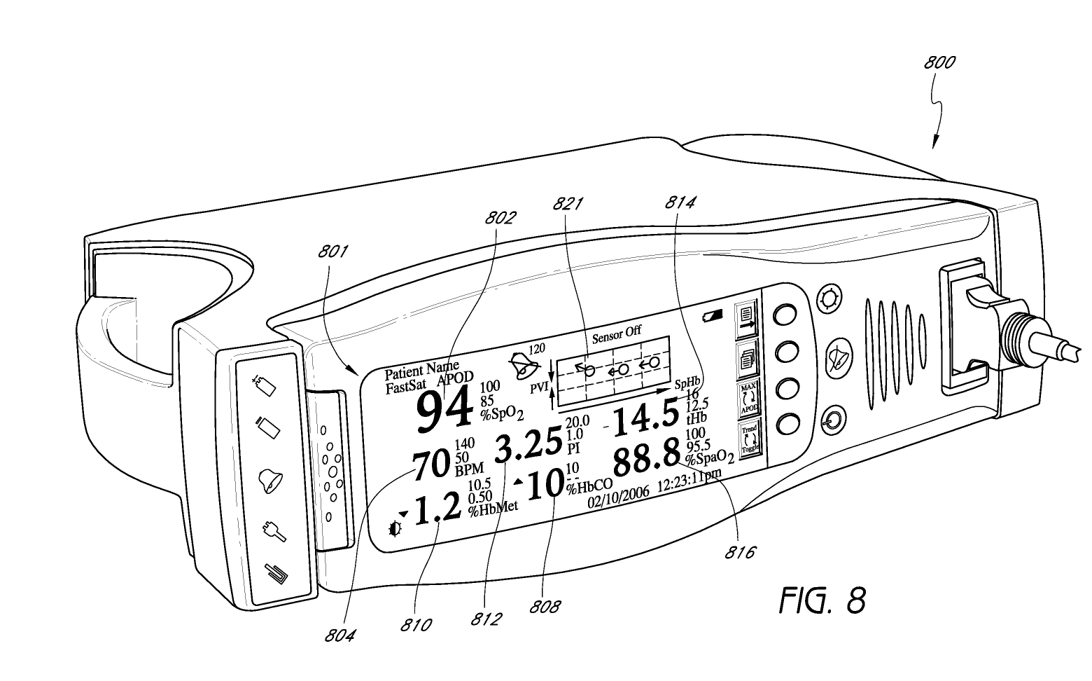

FIG. 8 illustrates an embodiment of a physiological measurement system 100 including a PVI vs. SpHb diagram 821 which graphically represents the relationship between PVI and SpHb. The relationship between PVI and SpHb can be an indication of various physiological conditions including, such as, for example, hydration, blood loss and heart failure. The PVI vs. SpHb diagram and indications of patient conditions are described in more detail below. Other parameters can also be used with diagram 821 for comparing parameter relationships in addition to PVI and/or SpHb or as alternatives to PVI and/or SpHb, including, such as, for example, blood pressure, PI, COHb MetHb, SpaO₂, SpO₂, pulse rate, or the like.

PVI is a measure of dynamic changes in the PI that occur during the respiratory cycle. PVI is calculated according to the following formula:

$$PVI = \frac{{PI}_{max} - {PI}_{min}}{{PI}_{max}} * 100 \% \qquad ( 1 )$$

where PI represents perfusion index. The calculation is accomplished by measuring changes in PI over a time interval where one or more complete respiratory cycles have occurred.

PI is the ratio of the pulsatile blood flow to the nonpulsatile or static blood in the peripheral tissue. PI can be determined based on the plethysmograph signal received using a non-invasive optical sensor, such as, for example, a sensor used in pulse oximetry. In general, pulse oximetry uses a red (R) light and infrared (IR) light. A constant amount of light (DC) from the signal of the pulse oximeter is absorbed by skin, bone and other static tissues including nonpulsatile blood. A variable amount of light (AC) is absorbed by the pulsating arterial inflow. PI can be determined using the following formula:

$$PI = \frac{AC}{DC} * 100 \% \qquad ( 2 )$$

In an embodiment, in order to calculate PI, the IR pulsatile signal is indexed against the non-pulstile IR signal and expressed as a percentage. In general the IR signal is used because it is less affected by changes in arterial saturation than the R signal.

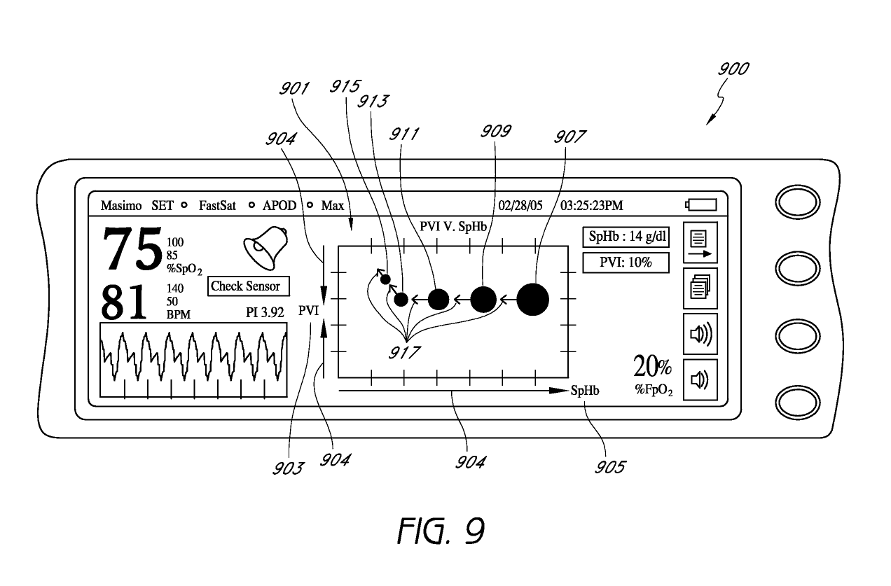

FIG. 9 illustrates an embodiment of a display 900 for a physiological measurement system. Display 900 includes a PVI/SpHb diagram 901. The diagram displays an indication of a patients condition by graphically representing the patients PVI vs. SpHb. The relationship between PVI and SpHb can indicate various patient conditions including, such as, for example, fluid levels, blood loss, and heart conditions, among others. The vertical axis 903 represents PVI levels. The horizontal axis 905 represents SpHb levels. Arrows 904 indicate where the optimal PVI and SpHb readings are generally located on the diagram. For example, an optimal reading would include a PVI measurement in the vertical middle of the diagram, and the optimal SpHb reading would be at the horizontal right side of the diagram.

Indicators 907-915 indicate the patient's status. Indicators of any shape can be used, for example, as displayed, a circle indicates the patient's status. A person of skill in the art will recognize from the disclosure herein, that other shapes can be used with the diagram, including, such as, for example, squares, diamonds, triangles, hexagons, or the like. In an embodiment, the size of the indicators 907-915 can indicate a length of time at that measurement or it can indicate an older or newer measurement. In an embodiment, older indicators can become lighter or darker in color or change to a different color or size. In an embodiment, the newest measurement or older measurements blink. Arrows 917 indicate trend information or information indicating the direction in which the measurements are moving. In an embodiment, instead of arrows, the shape of the indicator is used to indicate the direction in which the measurements are moving. For example, if triangles or chevrons are used, the triangles can be rotated to indicate the trend direction.

In general, a high PVI measurement can indicate hypovolemic state while a low PVI measurement can indicate hypervolemic state. Changes in the PVI trend can indicate that a patient is loosing fluids if the trend is increasing or receiving fluids if the trend is decreasing. A high total hemoglobin can indicate that a patient has sufficient hemoglobin in their blood. A low total hemoglobin can indicate that a patient does not have enough hemoglobin in their blood. Changes in the SpHb trend can indicate blood loss, if the SpHb is going down, or an increase in blood, such as by a transfusion, if the SpHb is going up. Other patient conditions can also be indicated based on the PVI v. SpHb diagram including, such as, for example, heart failure.

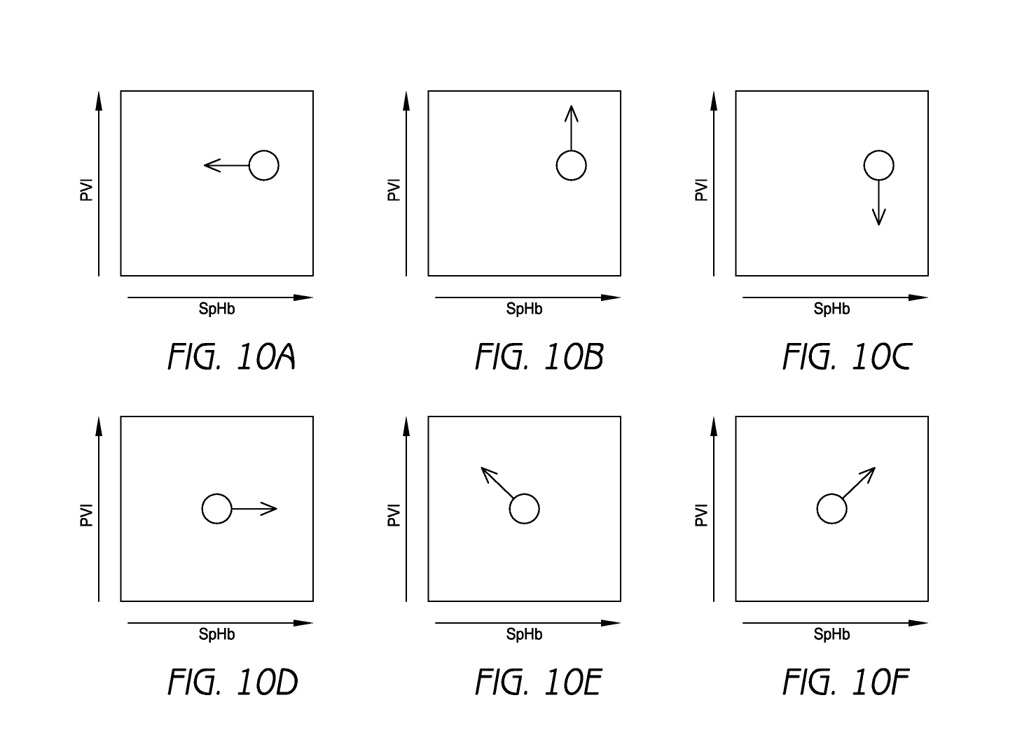

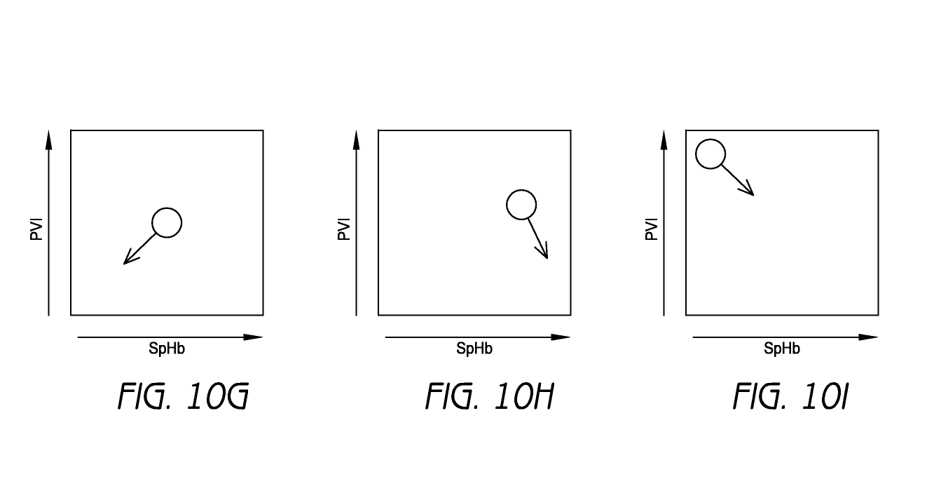

FIGS. 10A-10I illustrate screen shots of potential PVI vs. SpHb diagrams. Each display indicates a different condition which a patient can be experiencing. For example, FIG. 10A illustrates a patient with high but decreasing SpHb and a normal stable PVI measurement. This can indicate that the patient is bleeding but is euvolemic. FIG. 10B illustrates a patient with high stable SpHb with a normal but increasing PVI measurement. This can indicate that the patient is not getting enough fluid. FIG. 10C illustrates a patient with a high stable SpHb measurement and a normal but falling PVI measurement. This can indicate that the patient is getting too much fluid. FIG. 10D illustrates a normal and stable PVI reading and an increasing SpHb reading. This can indicate that the patient is receiving a blood transfusion. FIG. 10E illustrates a patient with decreasing SpHb and increasing PVI. This can indicate that a patient is loosing blood and not getting enough fluid. FIG. 10F illustrates a patient with increasing SpHb and increasing PVI. This can indicate that the patient is becoming hemoconcentrated or dehydrated. FIG. 10G illustrates a patient with decreasing SpHb and decreasing PVI. This can indicate that the patient is bleeding and/or the patient is receiving too much fluid. FIG. 10H illustrates a patient with increasing SpHb and decreasing PVI where the PVI reading is either still normal or is already low. This can indicate that the patient is suffering cardiac failure unrelated to excess volume. FIG. 10I illustrates a patient with a high but decreasing PVI and a low but increasing SpHb. This can indicate that the patient is receiving an effective blood transfusion. Other patient conditions will also be indicated by the PVI/SpHb graph as will be understood by a person of ordinary skill from the present disclosure. For example, as discussed above, during dialysis there can be an inflection point in the total hemoglobin concentration when too much fluid has been removed from a patient. Similarly, there will be an inflection point in the PVI measurement at the same time.

Although PVI and SpHb change within a patient, those changes may not exceed acceptable limits. Large changes may be acceptable as long as they remain within an acceptable range while small changes may indicate a serious problem if they move a patient's reading out of an acceptable range. In addition, large changes even in an acceptable range may indicate that a patient may be leaving the acceptable range, and thus that a problem is about to occur. In order to aid a care giver in determining whether changes in PVI are important, boundary indicators can be provided on a display. The boundaries can be based on empirically collected data from a large number of patients to determine a “normal” boundary, or it can be determined based on an individual patient's history. Additionally, the empirically obtained boundaries can be adjusted based on the patient's history or a condition of the patient or a treatment provided to the patient.

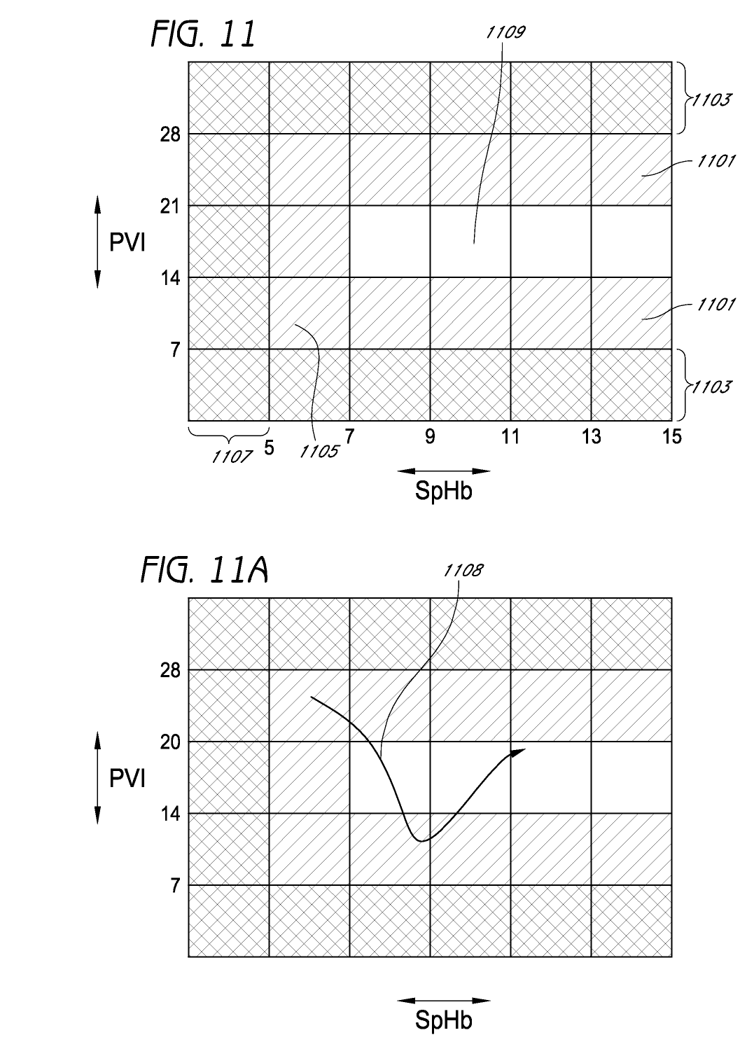

For example, in an embodiment as illustrated in FIG. 11, boundary lines are provided to illustrate acceptable ranges of PVI and SpHb. For example, areas 1101 illustrate areas of PVI measurements that may indicate that PVI measurements are becoming too high or too low and should be closely monitored. Areas 1103 may indicate that the patient's PVI readings are no longer within an acceptable range. Areas 1105 and 1107 likewise may indicate that SpHb readings are becoming or are too low. Area 1109 is an area where both SpHb and PVI are within normal or desired ranges. These boundary areas can be fixed or dynamic. For example, the boundaries can change based on a condition of the patient or a procedure being performed on a patient, such as, for example a blood transfusion or infusion of liquids. A boundary area change is illustrated, for example, in FIG. 11A. As illustrated in FIG. 11A, the PVI boundary line previously set at 21% is now set at 20%.

The usefulness of dynamic boundaries is illustrated, for example, in FIG. 11A. FIG. 11A illustrates a graph of a patient receiving a blood transfusion. An important consideration in providing a blood transfusion is to provide only the needed amount of blood so that a patient is not over-transfused. In order to aid a caregiver, the boundaries can be adjusted during a transfusion so that a caregiver can receive an indication that the patient has received a sufficient transfusion and that more transfusion is not necessary. The boundary change can be, for example, a narrowing of the acceptable boundary or an indicator line or set of lines indicating that the patient has received a sufficient transfusion. Boundaries can be indicated by color or shading, lines, dashed lines, color transition areas, or any other visual indications. For example, in an embodiment, areas 1107 and 1103 are red, areas 1101 and 1105 are yellow and area 1109 is green.

In an embodiment, the PVI/SpHb trend is also analyzed to determine a condition of a patient. For example, a quickly dropping PVI or SpHb measurement, though still in the acceptable range, may indicate that a patient is suffering a condition and may quickly have measurements outside of an acceptable range. Likewise, a trend may indicate that a sufficient transfusion has been received even though readings are not yet in an acceptable range because the trend indicates that the patient's readings will soon be within the acceptable range. In an embodiment, alarms are generated based on the trend and speed of changes observed. For example, in an embodiment, an alarm can be generated if the trend indicates significant changes even though the latest reading is still in an acceptable range. As another example, an alarm may sound for slow changes only when a reading is out of an acceptable range. Additionally, in addition to or as an alternative to alarms, the measurement can be used to administer blood transfusions, fluid infusions, or dialysis treatment, for example, using systems as described in FIGS. 4A-4D.

Conditional language used herein, such as, among others, “can,” “could,” “might,” “may,” “e.g.,” and the like, unless specifically stated otherwise, or otherwise understood within the context as used, is generally intended to convey that certain embodiments can include, while other embodiments may not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Depending on the embodiment, certain acts, events, or functions of any of the methods described herein can be performed in a different sequence, may be added, merged, or left out all together (e.g., not all described acts or events are necessary for the practice of the method). Moreover, in certain embodiments, acts or events may be performed concurrently, e.g., through multi-threaded processing, interrupt processing, or multiple processors or processor cores, rather than sequentially.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality may be implemented in varying ways for each particular application, but such implementation decisions should not be interpreted as causing a departure from the scope of the disclosure.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

While the above detailed description has shown, described, and pointed out novel features as applied to various embodiments, it will be understood that various omissions, substitutions, and changes in the form and details of the device or process illustrated may be made without departing from the spirit of the disclosure. As will be recognized, certain embodiments of the inventions described herein may be embodied within a form that does not provide all of the features and benefits set forth herein, as some features may be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A physiological monitoring system, the system comprising:
an optical sensor configured to:
transmit one or more wavelengths of optical radiation into a tissue site of a medical patient, and
detect the optical radiation after attenuation by pulsatile blood flowing within the tissue site so as to generate a sensor signal responsive to the detected optical radiation; and
a physiological monitor in communication with the optical sensor, the physiological monitor comprising one or more processors configured to:
derive hemoglobin values from the sensor signal, the hemoglobin values corresponding to values of hemoglobin concentration in the blood of the medical patient, and
generate a transfusion alarm based at least in part on one or more of the hemoglobin values, the transfusion alarm configured to recommend transfusing blood into the medical patient.

2. The system of claim 1, wherein the physiological monitor is further configured to generate the transfusion alarm in response to detecting one or more of the hemoglobin values being below a threshold hemoglobin value.

3. The system of claim 2, wherein a value of the hemoglobin threshold is based at least in part on one or more of an age of the medical patient, a gender of the medical patient, comorbidity of the medical patient, and a baseline hemoglobin value of the medical patient.

4. The system of claim 1, wherein the physiological monitor is further configured to generate the transfusion alarm in response to detecting a change in slope of a trend of two or more of the hemoglobin values.

5. The system of claim 1, wherein the physiological monitor is further configured to generate the transfusion alarm in response to detecting that an integration of one or more of the hemoglobin values exceeds an integration threshold.

6. A method of recommending a blood transfusion, the method comprising:
irradiating a tissue site of a medical patient using one or more wavelengths of light;
detecting the light after attenuation by blood flowing through the tissue;
generating a sensor signal based at least in part on the detected light;
deriving hemoglobin values from the sensor signal, the hemoglobin values corresponding to values of hemoglobin concentration in the blood of the medical patient;
processing one or more of the hemoglobin values to generate a transfusion alarm; and
outputting the transfusion alarm to a care provider.

7. The method of claim 6, wherein processing one or more of the hemoglobin values to generate the transfusion alarm comprises generating the transfusion detector in response to detecting one or more of the hemoglobin values being below a threshold hemoglobin value.

8. The method of claim 7, wherein a value of the hemoglobin threshold is based at least in part on one or more of an age of the medical patient, a gender of the medical patient, comorbidity of the medical patient, and a baseline hemoglobin value of the medical patient.

9. The method of claim 6, wherein processing one or more of the hemoglobin values to generate the transfusion alarm comprises generating the transfusion detector in response to detecting a slope of a trend of two or more of the hemoglobin values being below a slope threshold.

10. The method of claim 6, wherein processing one or more of the hemoglobin values to generate the transfusion alarm comprises generating the transfusion detector in response to detecting an integration value of one or more of the hemoglobin values exceeding an integration threshold.

11. A physiological monitoring system for monitoring dialysis patients, the system comprising:
an optical sensor comprising:
one or more emitters configured to transmit one or more wavelengths of light into a tissue site of a patient undergoing dialysis treatment, and
at least one detector configured to detect the light after attenuation by pulsatile blood flowing within the tissue site so as to generate a sensor signal responsive to the detected light; and
a physiological monitor configured to:
derive hemoglobin values from the sensor signal, the hemoglobin values corresponding to values of hemoglobin concentration in the blood of the patient, and
generate a dialysis alarm based at least in part on one or more of the hemoglobin values, the dialysis alarm configured to recommend adjusting the dialysis treatment to a care provider.

12. The system of claim 11, wherein the physiological monitor is further configured to generate the dialysis alarm in response to a slope of a trend of two or more of the hemoglobin values exceeding a slope threshold.

13. The system of claim 12, wherein a value of the slope threshold is based at least in part on one or more of an age of the medical patient, a gender of the medical patient, comorbidity of the medical patient, and a baseline hemoglobin value of the medical patient.

14. The system of claim 11, wherein the dialysis alarm is further configured to recommend stopping the dialysis treatment.

15. A method of monitoring dialysis patients, the method comprising:
irradiating tissue of a medical patient undergoing dialysis treatment using one or more wavelengths of light;
detecting the light after attenuation by blood flowing through the tissue;
generating a sensor signal based at least in part on the detected light;
deriving hemoglobin values from the sensor signal, the hemoglobin values corresponding to values of hemoglobin concentration in the blood of the medical patient;
processing one or more of the hemoglobin values to generate a dialysis alarm; and
using the dialysis alarm to recommend a change in the dialysis treatment.

16. The method of claim 15, wherein processing one or more of the hemoglobin values to generate a dialysis alarm comprises detecting a slope of a trend of two or more of the hemoglobin values decreasing below a slope threshold.

17. A physiological monitoring system for monitoring dialysis patients, the system comprising:
an optical sensor comprising:
one or more emitters configured to transmit one or more wavelengths of light into a tissue site of a patient undergoing dialysis treatment by a dialysis machine, and
at least one detector configured to detect the light after attenuation by pulsatile blood flowing within the tissue site so as to generate a sensor signal responsive to the detected light; and
a physiological monitor comprising one or more processors, the one or more processors configured to:
derive hemoglobin values from the sensor signal, the hemoglobin values corresponding to values of hemoglobin concentration in the blood of the patient,
generate a dialysis control signal based at least in part on one or more of the hemoglobin values, and
transmit the dialysis control signal to the dialysis machine, the dialysis control signal configured to cause the dialysis machine to adjust the dialysis treatment.

18. The system of claim 17, wherein the control signal is further configured to stop the dialysis treatment.

19. A physiological monitor sensor, the sensor comprising:
one or more emitters configured to transmit light through a tissue site of a medical patient; and
at least one detector configured to:
measure the light transmitted through the tissue site of the medical patient by the three or more emitters; and
generate at least one signal configured to be used by a processor to derive hemoglobin values corresponding to hemoglobin concentration in the blood of the patient and to generate a transfusion alarm based at least in part on one or more of the hemoglobin values.

20. The sensor of claim 19, wherein the transfusion alarm is generated in response to one or more of the hemoglobin values being below a threshold hemoglobin value.

21. The sensor of claim 19, wherein the transfusion alarm recommends transfusing blood into the medical patient.

22. The sensor of claim 19, wherein the sensor is capable of removable attachment to the tissue site of the medical patient.

23. A method of indicating a condition of a patient, the method comprising:
noninvasively determining an indication cardiac fluid responsiveness;
total hemoglobin; and
generating a display representing a relationship between the indication of plethysmograph variability and total hemoglobin.

24. The method of claim 23, wherein the determination of the indication of cardiac fluid responsiveness comprises a determination of one or more of plethysmograph variability, central venous pressure (CVP), pulsus paradoxus, pulmonary capillary wedge pressure (PCWP), stroke volume variation, pulse pressure variation, and systolic pressure variation (SPV).

25. The method of claim 23, further comprising activating an alarm when the relationship between the indication of plethysmograph variability and total hemoglobin meet predetermined criteria.

26. The method of claim 23, wherein generating a display comprises generating a graph with the indication of plethysmograph variability on a first axis of the graph and the indication of total hemoglobin on the second axis of the graph.

27. The method of claim 26, wherein the generating a display representing the relationship between the indication of plethysmograph variability and total hemoglobin further includes generating an indicator on the graph which indicates the relationship between the indication of the plethysmograph variability and total hemoglobin.

28. The method of claim 27, further comprising changing the indicator based on a length of time the indicator indicated the relationship between the indication of plethysmograph variability and total hemoglobin.

29. The method of claim 28, wherein changing the indicator comprises changing the size of the indicator.

30. The method of claim 27, wherein the indicator changes based on how recent the indicator indicated the relationship between the indication of plethysmograph variability and total hemoglobin.

31. The method of claim 30, wherein changing the indicator comprises fading the indicator.

32. The method of claim 30, wherein changing the indicator comprises changing the color of the indicator.

33. A physiological monitor display for indicating the condition of a patient, the physiological monitor comprising:

34. A noninvasive physiological monitoring system for displaying an indication of a physiological measurement of a patient comprising:
a sensor configured to emit light of at least two wavelengths onto tissue of a patient, the sensor further configured to detect light attenuated by the patient's tissue and generate a signal indicative of the detected attenuated light;
a monitor configured to receive the signal generated by the sensor and to determine at least two physiological parameters, the monitor including a display configured to graphically represent the relationship between the at least two physiological parameters.

35. The noninvasive physiological monitoring system of claim 34, wherein the at least two physiological parameters comprise total hemoglobin and plethysmograph variability.

36. The noninvasive physiological monitoring system of claim 34, wherein the display is configured to represent a current state of the relationship between the at least two physiological parameters.

37. The noninvasive physiological monitoring system of claim 36, wherein the display is configured to represent a trend direction associated with the current state of the relationship between the at least two physiological parameters.

38. The noninvasive physiological monitoring system of claim 36, wherein the display is configured to represent a previous state of the relationship between the at least two physiological parameters.
